(19) United States
(12) Patent Application Publication
(10) Pub. No.: US 2017/0196920 A1
(43) Pub. Date: Jul. 13, 2017

(54) Compositions and Related Methods For Treating Hyperadrenocorticism

(71) Applicant: James Marshall, III, Winter Park, FL (US)

(72) Inventor: James Marshall, III, Winter Park, FL (US)

(21) Appl. No.: 15/400,196

(22) Filed: Jan. 6, 2017

Related U.S. Application Data
(60) Provisional application No. 62/275,894, filed on Jan. 7, 2016.

(51) Int. Cl. A61K 35/747; A61K 31/4045; A61K 9/20; A61K 31/685; A61K 35/745; A61K 9/00; A61K 31/365

(52) U.S. Cl. CPC A61K 35/747; A61K 9/0053; A61K 31/4045; A61K 31/365; A61K 31/685; A61K 35/745; A61K 9/2009; A61K 9/2013; A61K 9/2054

(57) ABSTRACT

A composition for treating hyperadrenocorticism includes a therapeutically effective amount of a pharmaceutical dosage form including melatonin, lignan, phosphatidylserine, and at least one probiotic organism-containing material. The composition may be used to treat canine Cushing's disease, for example.

### CROSS-REFERENCE TO RELATED APPLICATION

This claims priority from U.S. provisional Application No. 62/275,894, filed Jan. 7, 2016 which is incorporated by reference in its entirety.

### FIELD

This relates to the field of dietary supplements and, more particularly, to dietary supplements that address hyperadrenocorticism.

### BACKGROUND

Hyperadrenocorticism is a condition characterized by the overproduction of glucocorticoids by the body. Cushing's disease, a common disease associated with hyperadrenocorticism, affects many mammals including dogs, cats, horses, and humans. Cushing's disease is caused by a tumor of the pituitary gland or adrenal gland. The tumor stimulates the production of glucocorticoids, such as cortisol and cortisone, causing hair loss, muscle weakness, skin problems, and lack of energy. Hyperadrenocorticism typically affects animals that are middle-aged or older. Some of the conventional treatments for hyperadrenocorticism use drugs that are expensive and have negative side effects.

### BRIEF SUMMARY

In view of the foregoing, it would be advantageous to treat hyperadrenocorticism with a composition that uses glucocorticoid-lowering ingredients. Such a composition includes melatonin, lignan, phosphatidylserine, and at least one probiotic organism-containing material.

The composition may be in a dosage form for oral delivery such as a pill or the like.

The probiotic organism-containing material may include both L. acidophilus and B. animalis.

The lignan may include 7-hydroxymatairesinol.

The therapeutically effective amount may include about 1-10 mg melatonin, about 15-40 mg lignan, about 80-100 mg phosphatidylserine, and about 150-200 mg of the at least one probiotic organism-containing material.

The amount of lignan in the dosage form may be at least three times greater than the amount of melatonin. The amount of phosphatidyl serine may be at least two times greater than the amount of lignan. The amount of the at least one probiotic organism-containing material may be at least two times greater than the amount of phosphatidyl serine.

The composition may be used to lower glucocorticoid levels in a patient by delivering a therapeutically effective amount of a dosage form including the composition to a patient in need thereof. The composition is particularly useful for ameliorating symptoms of canine Cushing's disease.

### DETAILED DESCRIPTION OF EXAMPLE EMBODIMENTS

This disclosure describes preferred embodiments, but not all possible embodiments of the compositions and methods. Where a particular feature is disclosed in the context of a particular embodiment, that feature can also be used, to the extent possible, in combination with and/or in the context of other embodiments. The compositions and methods may be embodied in many different forms and should not be construed as limited to only the embodiments described here.

A composition for treating hyperadrenocorticism is first described. The composition generally includes melatonin, lignan, phosphatidylserine, and at least one probiotic organism-containing material.

“Melatonin” refers to the hormone melatonin and derivatives thereof. Melatonin is effective to lower the cortisol level in the body.

A “lignan” is a compound from the class of compounds called lignans. Lignans also lower the cortisol level in the body. Lignans are phenolic compounds that are often found in plants, but are also found in mammals. Examples of plant-derived lignans include, but are not limited to, arctigenin, pinoresinol, lariciresinol, isolariciresinol, matairesinol, medioresinol, hydroxymatairesinol, syringaresinol, and sesamin. Examples of mammalian lignans include enterdiol and enterolactone. It has been reported that that bacteria in the body metabolize some plant-derived lignans to mammalian lignans.

7-hydroxymatairesinol (7-HMR) is a particular example of a lignan that may be used in the composition. 7-HMR is derived from Picea abies (Norway Spruce).

Phosphatidylserine is a phospholipid found naturally in the body's cell membranes. Phosphatidylserine may reduce cortisone levels. Although not intending to be bound by theory, phosphatidyl serine may also assist with the absorption of melatonin in the gastrointestinal tract because it can form liposome-type vesicles that may carry melatonin therein.

The “at least one probiotic organism-containing material” includes one or more organisms that benefit the metabolism of melatonin, lignan, and/or phosphatidylserine in the body. Examples of probiotic organisms include, but are not limited to, bacteria such as Lactobacillus acidophilus (L. acidophilus) and Bifidobacterium animalis (B. animalis). L. acidophilus bacteria occur naturally in the human digestive tract and are sometimes used as an additive in dairy products. B. animalis bacteria are found in intestines of many mammals.

A “therapeutically effective amount” is an amount effective to achieve a desired therapeutic benefit, such as an amount effective to prevent, alleviate, ameliorate, or treat the underlying causes and/or symptoms of the physiological condition being treated. For some uses of the composition, the therapeutically effective amount may be an amount effective to lower a glucocorticoid level in the patient being treated and/or alleviate one or more of the symptoms of Cushing's disease.

A therapeutically effective amount range may be often 1-1,000 mg/day, including 1-25 mg/day, 25-50 mg/day, 50-75 mg/day, 75-100 mg/day, 100-150 mg/day, 150-200 mg/day, 200-250 mg/day, 250-300 mg/day, 300-350 mg/day, 350-400 mg/day, 400-450 mg/day, 450-500 mg/day, 500-550 mg/day, 550-600 mg/day, 600-650 mg/day, 650-700 mg/day, 700-750 mg/day, 750-800 mg/day, 800-850 mg/day, 850-900 mg/day, 900-950 mg/day, 950-1,000 mg/day. Higher doses (1,000-3,000 mg/day) might also be effective. The weight in mg is often calibrated to the body weight of the patient in kg, thus these example doses may also be written in terms of mg/kg of body weight per day.

In practice, the therapeutically effective amount may vary depending on numerous factors associated with the patient, including age, weight, height, severity of the disorder, administration technique, and other factors. The therapeutically effective amount administered to a patient may be determined by medical personnel taking into account the relevant circumstances.

The therapeutically effective amount may be determined or predicted from empirical evidence. Specific dosages may vary according to numerous factors and may be initially determined on the basis of in vitro, cell culture, and/or in vivo studies. The amount of glucocorticoids the patient may provide useful guidance in determining a therapeutically effective amount for administration.

The composition may be administered as a single dose or as part of a dosage regimen. For a dosage regimen, the therapeutically effective amount adjustable dose to dose to provide a desired therapeutic response.

Multiple doses may be administered at a predetermined time interval and subsequent doses may be proportionally reduced or increased, depending on the situation.

The composition may be blended with one or more pharmaceutically acceptable excipients useful for making the composition into a pharmaceutically acceptable dosage form.

Exemplary excipients include, but are not limited to, carriers, diluents, disintegrants, emulsifiers, solvents, processing aids, buffering agents, colorants, flavorings, solvents, coating agents, binders, carriers, glidants, lubricants, granulating agents, gelling agents, polishing agents, suspending agent, sweetening agent, anti-adherents, preservatives, emulsifiers, antioxidants, plasticizers, surfactants, viscosity agents, enteric agents, wetting agents, thickening agents, stabilizing agents, solubilizing agents, bioadhesives, film forming agents, emollients, dissolution enhancers, dispersing agents, or combinations thereof.

Suitable lubricants include, but are not limited to, stearic acid and magnesium stearate.

Suitable binders include, but are not limited microcrystalline cellulose and other pharmaceutical acceptable binders.

Suitable glidants include, but are not limited to, silicon dioxide and other pharmaceutically acceptable glidants.

Suitable diluents include, but are not limited to, dicalcium phosphate and other pharmaceutically acceptable diluents.

Suitable disintegrants include, but are not limited to, croscarmellose sodium and other pharmaceutically acceptable disintegrants.

The dosage form, such as a tablet, capsule, or the like, may be coated to aid in swallowing, to mask the taste of the ingredients, improve appearance, and/or protect the dosage form from moisture. The coating may be applied using conventional coating techniques, such as, for example, spray coating, bed coating, or the like. An example of a suitable coating is pharmaceutical glaze, which is a shellac-based coating material. Pharmaceutical glaze is typically provided as a solution of the shellac-based coating material dissolved in an alcohol such as ethanol.

The composition may he used to treat hyperadrenocorticism by administering it to an animal in need thereof.

Some administration techniques involve administering the composition as a unit dosage form. For example, unit dosage forms suitable for oral administration include solid dosage forms, such as powders, granules, pills (tablets, capsules, caplets), suppositories, and sachets. They also include liquid dosage forms, such as elixirs, syrups, suspensions, sprays, gels, lotions, creams, foams, ointments, salves, solutions, tinctures, and emulsions.

A method of treatment includes lowering a glucocorticoid level of the animal patient by administering to an animal in need thereof a composition comprising a therapeutically effective amount of melatonin, lignan, phosphatidylserine, and at least one probiotic organism-containing material.

The dose of the composition administered to the animal may vary depending on the animal's body weight. In a particular example, the dose may be about 1-10 mg melatonin plus about 15-40 mg lignan plus about 80-100 mg phosphatidyl serine per 30 lbs. (13.6 kg) body weight per day.

in a particular example of the composition, the dosage form is a tablet and the therapeutically effective amount includes about 1-10 mg melatonin, about 15-40 mg lignan, about 80-100 mg phosphatidyl serine, and about 150-200 mg of the at least one probiotic organism-containing material. The tablet may be administered orally.

In another example of the composition, the amount of lignan is at least three times greater than the amount of melatonin and the amount of phosphatidyl serine is at least two times greater than the amount of lignan. The amount of the probiotic ingredient(s) may be at least two times greater than the amount of phosphatidyl serine.

In another particular example of the composition, the dosage form is a tablet and the therapeutically effective amount includes about 3.6 mg melatonin, about 24 mg lignan, about 90 mg phosphatidyl serine, and about 180 mg of at least one probiotic organism-containing material.

The composition is particularly advantageous for treating animals, such as dogs, with Cushing's disease. Dogs with Cushing's disease experience symptoms such as panting, hunger, thirst, muscle weakness, lack energy, potbelly abdomen, and hair loss among others The composition may alleviate one or more of these symptoms, improving the health of the dog.

In some cases, it may be desirable to administer the composition to the an during the evening or night and in combination with food.

The composition may prepared by combining a therapeutically effective amount of melatonin, phosphatidylserine, and at least one probiotic organism-containing material into a dosage form suitable for oral delivery. If the dosage form is a tablet, these ingredients and any excipients may be compressed together to form the tablet. A coating may be applied to enhance the tablet's mechanical properties and/or taste.

The melatonin, lignan, phosphatidylserine, and at least one probiotic organism containing may be combined as solid ingredients such as a granulation or powder.

### EXAMPLE

This section provides an example of the composition. The scope of the possible aspects and embodiments is not limited to what this example teaches.

A compressed tablet was prepared compressing a mixture of the ingredients of Table 1 into a monolithic tablet. The monolithic tablet was administered to a dog with Cushing's disease at a dose of 1 tablet per 30 lbs. (13.6 kg) body weight per day in the evening. The dog's symptoms improved during the treatment.

TABLE 1
Tablet Dosage Form Example

| Ingredient | Mass (milligrams) | |
|---|---|---|
| Melatonin | 3.6 | mg |
| Lignan (7-HMR) | 24 | mg |
| Phosphatidylserine | 90 | mg |
| B. animalis | 150 | mg |
| L. acidophilus | 30 | mg |
| Microcrystalline | 131 | mg |
| cellulose | | |
| Dicalcium phosphate | 85 | mg |
| Stearic acid | 65 | mg |
| Croscarmellose sodium | 25 | mg |
| Magnesium Stearate | 10 | mg |
| Silicon dioxide | 5 | mg |
| Pharmaceutical glaze | | |
| (shellac and povidone) | | |

That which is claimed is:

1. A composition for treating hyperadrenocorticism comprising a solid oral pharmaceutical dosage form effective for lowering a glucocorticoid level of a patient, the oral pharmaceutical dosage form including 2-6 mg melatonin, 22-26 mg 7-hydroxymatairesinol, 88-92 mg phosphatidylserine, 148-152 mg B. animalis, and 28-32 mg L. acidophilus.

2. The composition of claim 1, wherein the solid oral pharmaceutical dosage form is a compressed tablet and the melatonin, 7-hydroxymatairesinol, phosphatidyl serine, B. animalis and L. acidophilus are powders.

3. The composition of claim 1, further comprising 83-87 mg dicalcium phosphate, 63-67 mg stearic acid, 23-27 mg croscarmellose sodium, 8-12 mg magnesium stearate, and 3-7 mg silicon dioxide.

4. The composition of claim 1, wherein the solid oral pharmaceutical dosage form is a compressed tablet and further comprising 83-87 mg dicalcium phosphate, 63-67 mg stearic acid, 23-27 mg croscarmellose sodium, 8-12 mg magnesium stearate, and 3-7 mg silicon dioxide, wherein the melatonin, 7-hydroxymatairesinol, phosphatidyl serine, B. animalis, L. acidophilus, dicalcium phosphate, stearic acid, croscarmellose sodium, magnesium stearate, and silicon dioxide are powders.

5. A composition for treating hyperadrenocorticism, the composition comprising a therapeutically effective amount of a pharmaceutical dosage form including melatonin, lignan, phosphatidylserine, and at least one probiotic organism containing material.

6. The composition of claim 5, wherein melatonin, lignan, phosphatidylserine, and at least one probiotic organism-containing material are combined in a dosage form for oral delivery.

7. The composition of claim 6, wherein the dosage form is a pill.

8. The composition of claim 5, wherein the at least one probiotic organism-containing material includes both L. acidophilus and B. animalis.

9. The composition of claim 5, wherein the lignan includes 7-hydroxymatairesinol.

10. The composition of claim 5, wherein the therapeutically effective amount includes about 1-10 mg melatonin, about 15-40 mg lignan, about 80-100 mg phosphatidylserine, and about 150-200 mg of the at least one probiotic organism-containing material.

11. The composition of claim 5, wherein the therapeutically effective amount includes about 3.6 mg melatonin, about 24 mg lignan, about 90 mg phosphatidylserine, and about 180 mg of the at least one probiotic organism-containing material.

12. The composition of claim 5, wherein the therapeutically effective amount is effective for lowering a glucocorticoid level in a patient.

13. The composition of claim 5, wherein the therapeutically effective amount is effective for treating Cushing's disease.

14. The composition of claim 5, wherein the amount of lignan in the dosage form is at least three times greater than the amount of melatonin, the amount of phosphatidyl serine is at least two times greater than the amount of lignan and the amount of the at least one probiotic organism-containing material is at least two times greater than the amount of phosphatidyl serine.

15. A process of making a composition for treating hyperadrenocorticism, the process comprising combining a therapeutically effective amount of melatonin, lignan, phosphatidylserine, and at least one probiotic organism-containing material into a pharmaceutical dosage form suitable for oral delivery.

16. The process of claim 15, further comprising compressing the melatonin, lignan, phosphatidylserine, and at least one probiotic organism-containing material into a tablet and the dosage form suitable for oral delivery is a compressed tablet.

17. The process of claim 15, wherein the melatonin, lignan, phosphatidylserine, and at least one probiotic organism-containing material are combined as solid ingredients.

18. The process of claim 15, wherein the at least one probiotic organism-containing material includes both L. acidophilus and B. animalis.

19. The process of claim 15, wherein the lignan includes 7-hydroxymatairesinol.

20. The process of claim 15, wherein the therapeutically effective amount includes about 1-10 mg melatonin, about 15-40 mg lignan, about 80-100 mg phosphatidylserine, and about 150-200 mg of the at least one probiotic organism-containing material.

21. The process of claim 15, wherein the therapeutically effective amount is effective for lowering a glucocorticoid level in a patient.

22. A method of treating hyperadrenocorticism in an animal, the method comprising lowering a glucocorticoid level of the animal patient by administering to an animal in need thereof a composition comprising a therapeutically effective amount of melatonin, lignan, phosphatidylserine, and at least one probiotic organism-containing material.

23. The method of claim 22, wherein the composition is administered orally.

24. The method of claim 22, wherein the composition is administered as a pill dosage form.

25. The method of claim 22, wherein the at least one probiotic organism-containing material includes both L. acidophilus and B. animalis.

26. The method of claim 22, wherein the therapeutically effective amount includes about 1-10 mg melatonin, about 15-40 mg lignan, about 80-100 mg phosphatidylserine, and about 150-200 mg of the at least one probiotic organism-containing material.

27. The method of claim 22, wherein the dosage form comprises 1-10 mg melatonin, 15-40 mg lignan, and 80-100 mg phosphatidyl serine and is administered per 13 kg of the animal's body weight per day.
